(19) United States
(12) Patent Application Publication
(10) Pub. No.: US 2023/0352181 A1
(43) Pub. Date: Nov. 2, 2023

(54) AI-ENABLED EXPEDITED RISK ASSESSMENT OF PATIENTS WITH CHEST PAIN

(71) Applicant: Morteza Naghavi, Long Beach, CA (US)

(72) Inventor: Morteza Naghavi, Long Beach, CA (US)

(21) Appl. No.: 18/167,852

(22) Filed: Feb. 11, 2023

Related U.S. Application Data
(63) Continuation-in-part of application No. 17/657,754, filed on Apr. 2, 2022, now Pat. No. 11,610,686.
(60) Provisional application No. 63/414,546, filed on Oct. 9, 2022.

(51) Int. Cl. G16H 50/30; G16H 30/40; G16H 40/20; G16H 50/20

(52) U.S. Cl. CPC G16H 50/30; G16H 30/40; G16H 40/20; G16H 50/20

(57) ABSTRACT

Risk assessment systems and methods for patients with chest pain include, in some embodiments, an assessor, a computed tomography (CT) scanner, an electrocardiogram device for providing electrocardiogram data, and an enzyme analyzer for analyzing the patient’s blood. A computer enabled risk calculator categorizes the patients as low, intermediate, or high risk. The computer enabled risk calculator, using electrocardiogram data, blood analyzer, patient’s age, other risk factors, and history, automatically generates orders for low and intermediate risk patients to undergo a CT scan. A CAC analyzer provides a CAC score and/or a cardiovascular volumetry index based on CT scan results. A risk score based on electrocardiogram, blood analyzer and patient’s age, other risk factors and history of symptoms, the CAC score and/or cardiovascular volumetry index is generated. Patients that are automatically assessed as being very low risk based on the risk score are recommended for discharge from the emergency room.

### BACKGROUND

### Related Applications

This application is as continuation-in-part of co-pending U.S. Pat. Application 17/657,754, filed on Apr. 2, 2022, which is hereby incorporated herein in its entirety by reference. This application claims priority to U.S. Provisional Pat. Application 63/414,546 filed on Oct. 9, 2022, which is hereby incorporated herein in its entirety by reference.

### FIELD

The inventive subject matter is generally directed towards systems and methods for efficient risk assessment of patients with chest pain. In particular, embodiments of the invention relate to system and methods for recommending discharge of a patient based, at least in part, on Al-enabled determination of CAC scores and a cardiac volumetry index.

### DESCRIPTION OF RELATED ART

Coronary Artery Calcium (CAC) imaging proves the presence of coronary artery disease with near 100% specificity and predicts the risk of serious coronary events. Studies have shown that CT calcium score facilitate risk prediction and is more predictive than any other single biomarker. Adding CAC score to traditional risk factors improves AUCs (Area Under (Receiver Operating Characteristic) Curve) for risk prediction.

According to the Centers for Disease Control (CDC), Americans make 130 million visits to the Emergency Department (ED) every year. About 8% to 10% of these visits are symptoms of acute chest pain-up to 13 million visits a year. Except for trauma injuries, chest pain is one of the most common reasons for going to the ED. Persons experiencing chest pain should in general call emergency medical services or get to the ED as quickly as possible for rapid evaluation and treatment. However, the majority of ED patients who present with suspected Acute Coronary Syndrome (ACS) fall below the 1% risk threshold of a 30-day Major Adverse Coronary Event (MACE). About 50% of patients with chest pain symptoms-across all age groups-will have a diagnosis of nonspecific chest pain, unrelated to any cardiac condition.

ED providers are challenged to safely and responsibly identify as well as classify patients who present with symptoms of chest pain so they can avoid the risk of inadvertently discharging any patient at risk for a 30-day MACE. The HEART Scoring System for Chest Pain, recommended by the American Heart Association, has shown to be effective in predicting outcomes for patients with chest pain. The HEART Score assigns 0-2 points for each of five categories. FIG. 1 shows exemplary HEART Score information. As shown in the table in FIG. 10, the percentage of low and intermediate risk ED patients with chest pain is more than 50%.

Although the HEART Score includes an ECG and troponin result, which can indicate a very low risk of a 30-day MACE, most clinicians are not comfortable discharging a patient to home.

For coronary artery disease certain screening and diagnostic tools are known, such as coronary artery calcium (CAC) score and coronary CT angiography. And for predicting atrial fibrillation (AF) and heart failure (HF) it is known to use, for example, CHARGE-AF and brain natriutic peptide (BNP). CHARGE AF is an epidemiological risk calculator. BNP is more precise than CHARGE-AF but it is not specific to left atrial and ventricular function.

AF is the most common sustained arrhythmia and is associated with an increased risk of stroke and cardiovascular mortality. In the United States, at least 3 to 6 million people have AF. It is predicted that the number of AF patients will increase to over 12 million cases by 2030, imposing a significant economic burden with projected healthcare expenses of $260 million. In Europe, prevalent AF in 2010 is about 9 million among individuals older than 55 years and is expected to reach 14 million by 2040. It is estimated that by 2050 at least in 72 million individuals in Asia will be diagnosed with AF, and about 3 million with AF-related strokes. This presents a public health crisis especially for the growing elderly population in coming decades.

Medicare services costs are significantly higher among AF patients than non-AF patients; therefore early treatment is critical to limit the disease burden imposed by AF. The adverse social and public health effects of HF are even worse. It is estimated that by 2030, more than 8 million Americans will have HF. And the total direct medical costs of HF are expected to rise from $21 billion to $53 billion.

The 5-year survival rates of AF are of concern. Without proper treatment, 51% of AF patients will die within five years. Although the economic burden posed by AF and HF are critical in light of the increasing healthcare costs, early detection tools and preventive interventions for pre-AF and pre-HF patients are currently unavailable. One report shows that 96,860 strokes occurred within 1 year among patients with AF, with an associated total direct lifetime cost of nearly $8 billion. Of these costs, $2.6 billion in direct costs are incurred during the first year after the stroke.

HF poses an even greater threat to US healthcare system. Given the rising rates of hospitalization and rehospitalization, HF is associated with a significant cost burden. Approximately 1% to 2% of the total US health care budget is spent on HF, and half of that is attributable to late diagnosis leading to inpatient admissions for HF. This challenge presents a great opportunity to make an impact on the healthcare system by early detection and interventions of subclinical HF and AF. Currently, BNP and CHARGE-AF are the only available tools for early detection of high-risk patients for AF and HF. A combination of high-risk CHARGE-AF and a 7-day ECG patch has been reported. CHARGE-AF is an epidemiological risk calculator that can be useful as a population based measure, but it is not preferred as applied to individual patients as needed in a physician’s office. A more direct assessment is preferred such as by imaging the cardiac chambers where AF happens.

It is known to calculate a CHARGE-AF score: 0.508 × age (5 year increments) + 0.248 × height (10 cm increments) + 0.115 × weight (15 Kg increments) + 0.197 × systolic blood pressure (20 mm Hg increments) - 0.101 × diastolic blood pressure (10 mm Hg increments) + 0.359 × current smoker + 0.349 × antihypertensive medication + 0.237 × diabetes + 0.701 × congestive heart failure + 0.496 × myocardial infarction. See, “Simple risk model predicts incidence of atrial fibrillation in a racially and geographically diverse population: the CHARGE-AF consortium.” J Am Heart Assoc. 2013 Mar 18;2(2):e000102. doi: 10.1161/JAHA.112.000102. PMID: 23537808; PMCID: PMC3647274.

It is known to use manual measurements of left ventricle chamber in a single-slice to predict HF. However, rapid and accurate acquisition of whole heart volume parameters is challenging. Even though semi-automated delineation and quantification of cardiovascular structures can be useful in CT images, currently known methods still require a significant degree of manual modifications, which is time-consuming and may increase inter-/intra-observer variability.

Over a billion will die from cardiovascular disease, most being unaware of their risk die prematurely. The need for early detection of pre-symptomatic cardiovascular disease is unmet. So is the need for inexpensive, reliable and scalable technology. CAD can be assessed by a CAC scan. Blood pressure and lipid levels are easily measured, but currently, other cardiac anatomical risk factors cannot be quantified without complex testing.

Currently, no screening tool is available for detecting individuals at high risk of AF and/or HF. Despite the critical need no biomarker is currently available to identify individuals at high risk for AF or HF and their complications. An ideal biomarker would help to detect individuals at high risk for both AF and HF.

In view of the above, there is a long-felt need in the healthcare industry to improve the workflow in health care settings as well as to reduce patient wait times using various systems and methods. Embodiments of the systems and methods disclosed here address these and other needs in the relevant art.

### SUMMARY OF ILLUSTRATIVE EMBODIMENTS

Data integration projects face challenges bringing together the right expertise. There are pipeline behaviors that can be common across multiple pipelines. However, under known methods, typically pipeline behaviors are not developed once and made reusable by different pipelines. Some embodiments disclosed herein address the need for scale where dozens of projects may be concurrently active.

In one aspect, the invention is directed to a system for assessing a patient with chest pain, the patient presenting at an emergency department of a hospital. In one embodiment, the system includes an assessor configured to assess a history of cardiovascular risk factors and symptoms of chest pain of a patient; an electrocardiogram device for obtaining electrocardiogram related data from the patient; an enzyme analyzer configured to determine troponin enzyme levels associated with the patient; a CT scan analyzer configured to provide a CAC score and a cardiovascular volumetry risk index based on CT scan images of the patient; a computer enabled risk calculator configured to: (a) generate a first risk score for the patient based on electrocardiogram, troponin level, age of the patient, other risk factors, and history of symptoms; (b) based at least in part on the first risk score, assign to the patient a risk category selected from the group of risk categories comprising: low, intermediate, and high; (c) generate an order for the patient to undergo a non-contrast cardiac CT scan if the risk category assigned to the patient is low or intermediate; (d) based at least in part on a CAC score and a cardiovascular volumetry risk index determined by the CT scan analyzer, determine a second risk score for the patient; and (d) based at least in part of the second risk score, recommend discharge of the patient from the emergency department if the second risk score is below a predetermine threshold.

In some embodiments, artificial intelligence is used to calculate a CAC score. In certain embodiments, the artificial intelligence includes detecting features from at least one or more portions of the received CT results, electrocardiogram related data, and troponin enzyme level data that fall within each of one or more temporal windows; identifying patterns in the detected features based on one or more of the following models: a classification model and a regression model; and using the identified patterns, calculate, a probability of whether the identified patterns correspond to a CAC score of a patient.

In another aspect, the invention concerns a method of expediting the risk assessment of a patient presenting with chest pain. In one embodiment, the method includes determining a first score based at least in part on data associated with the age and a troponin level of the patient; if the first score is below a predetermined risk level, obtaining a CAC score and a cardiovascular volumetry risk index associated with the patient; and based on the CAC score and the cardiovascular volumetry risk index, making a recommendation associated with discharging the patient.

In some embodiments, determining a first score includes determining a HEART score. In certain embodiments, the predetermined risk level is a HEART score less than 7. In one embodiment, obtaining a cardiovascular volumetry risk index includes obtaining an estimate of left ventricle volume. In some embodiments, obtaining an estimate of left ventricle volume includes using an artificial intelligence (Al) enabled volume calculator. In certain embodiments, using an AI enabled calculator includes using non-contrast enhanced CT scan images as input to the AI enabled calculator.

In yet another aspect of the invention, the invention is directed to a system for facilitating assessment of patients presenting with chest pain. In one embodiment, the system includes a clinical predictor configured to determine a first risk score associated with the patient; a CAC analyzer configured to provide a CAC score and a cardiovascular volumetry risk index based on CT scan images associated with the patient; and a risk calculator configured to: (a) determine a second risk score based on the CAC score and the cardiovascular volumetry risk index; and (b) based at least in part on the second risk score, to automatically provide a recommendation on whether to discharge the patient.

In some embodiments, the clinical predictor is configured to determine a first risk score based at least in part on a HEART score. In certain embodiments, the clinical predictor is configured to determine the first risk score based at least on one or more of: age, blood pressure, cholesterol levels, smoking status, and family history. In one embodiment, the clinical predictor is configured to determine the first risk score based at least on one or more of: patient’s age, electrocardiogram data, and patient troponin levels.

In some embodiments, the CT scan images include non-contrast enhanced CT scan images. In certain embodiments, the CAC analyzer is configured to use artificial intelligence (Al) to provide the CAC score and/or the cardiovascular volumetry index. In one embodiment, the AI is configured to provide the CAC score and/or the cardiovascular volumetry index based, at least in part, on analysis of non-contrast enhanced CT scan images associated with the patient. In some embodiments, the CAC analyzer is configured to provide the cardiovascular volumetry risk index by, at least in part, obtaining an estimate of left ventricle volume. In certain embodiments, using non-contrast enhanced CT scan images are used as input to the AI enabled calculator.

Yet another aspect of the invention concerns a method of assessing a patient presenting with chest pain. In one embodiment, the method can include assessing the patient’s health history and heart health symptoms; scanning the patient’s heart using computed tomography (CT); storing the CT results to a computer file; scanning the patient’s cardiac health using an electrocardiogram device;

storing the electrocardiogram generated data resulting from said scanning, the data comprising cardiac health data; analyzing the patient’s blood and obtaining one or more troponin enzyme levels of the patient’s blood; storing the troponin enzyme level data resulting from said testing; using a computer enabled risk calculator categorize the patient into low, intermediate, or high risk category, wherein the risk calculator is configured to categorize the patient, based at least in part on the electrocardiogram data, troponin levels, patient’s age, other risk factors, and patient history; automatically generating orders for a patient to undergo a CT scan if the patient is categorized in low or intermediate risk category; using a CAC analyzer to analyze the CT scan results and provide a CAC score and a cardiovascular volumetry index; using a computer enabled risk calculator to generate a risk score based on the CAC score and the cardiovascular volumetry index; assessing the patient automatically as being very low risk based on the risk score; and recommending discharge of the very low risk patient from the emergency room, thereby lowering unnecessary prolonged ER stay time for the patient.

In some embodiments, using a CAC analyzer includes using artificial intelligence (Al) to provide the CAC score and/or the cardiovascular volumetry index. In certain embodiments, using AI includes using non-contrast enhanced CT scan images as input to the AI. In one embodiment, using AI to provide the cardiovascular volumetry index includes estimating a volume of a left ventricle.

In another aspect, the invention concerns a method of assessing a patient’s risk of pulmonary embolism (PE). In one embodiment, the method includes assessing the patient’s health history and evaluating clinical likelihood of PE; measuring the patient’s D-dimer; automatically generating orders for the patient to undergo a CT scan if the patient is categorized in a low or an intermediate risk category; using an Al-enabled cardiovascular volume calculator to obtain a right ventricle (RV) volume and a left ventricle (LV) volume; determining a RV volume to LV volume ratio; and determining a PE risk based at least in part on the RV volume to LV volume ratio. In some embodiments, the CT scan includes a non-contrast enhanced CT scan.

Additional features and advantages of the embodiments disclosed herein will be set forth in the detailed description that follows, and in part will be clear to those skilled in the art from that description or recognized by practicing the embodiments described herein, including the detailed description which follows, the claims, as well as the appended drawings.

Both the foregoing general description and the following detailed description present embodiments intended to provide an overview or framework for understanding the nature and character of the embodiments disclosed herein. The accompanying drawings are included to provide further understanding and are incorporated into and constitute a part of this specification. The drawings illustrate various embodiments of the disclosure, and together with the description explain the principles and operations thereof.

### BRIEF DESCRIPTION OF THE DRAWINGS

A more complete understanding of the embodiments, and the attendant advantages and features thereof, will be more readily understood by references to the following detailed description when considered in conjunction with the accompanying drawings wherein:

FIG. 1 is a flowchart of a method of determining a risk of a patient for an adverse health condition according to embodiments of the invention disclosed herein.

FIG. 2 is a block diagram of a system for determining a risk of patient for an adverse health condition according to embodiments of the invention disclosed here.

FIG. 3 is a flowchart of a method of generating an artificial intelligence model for facilitating determining a risk of a patient for an adverse health condition according to embodiments of the invention disclosed here.

FIG. 4 is a flowchart of a method of applying an artificial intelligence model for facilitating determining a risk of patient for an adverse health condition according to embodiments of the invention disclosed here.

FIGS. 5A-5C are images illustrating examples of segmentations for cardiovascular structure volume measurements performed by, and according to, embodiments of the inventive methods and systems disclosed herein.

FIG. 6 is a graph showing cumulative incidence of HF by LV volume as performed by certain embodiments of the systems and methods disclosed herein.

FIG. 7 is a graph showing cumulative incidence of HF by LV volume quartiles as performed by certain embodiments of the systems and methods disclosed herein.

FIG. 8 is a graph showing cumulative incidence of AF by LA volume quartiles as performed by certain embodiments of the systems and methods disclosed herein.

FIG. 9 is a graph showing cumulative incidence of AF by LA volume as performed by certain embodiments of the systems and methods disclosed herein.

FIG. 10 is a table of a prior art scoring system.

FIG. 11 is a table of another prior art scoring system.

FIG. 12 is a flowchart of a method of risk assessment according to embodiments of the invention.

FIG. 13 is a block diagram of a system for risk assessment of symptomatic patients according to embodiments of the claimed subject matter.

FIG. 14 is a block diagram of another system for risk assessment of symptomatic patients according to embodiments of the claimed subject matter.

FIG. 15 is a flowchart of a method of risk assessment for persons presenting with chest pain according to embodiments of the invention.

FIG. 16 is a flowchart of a method of assessing a patient’s risk of pulmonary embolism according to embodiments of the invention.

### DETAILED DESCRIPTION OF ILLUSTRATIVE EMBODIMENTS

The specific details of the single embodiment or variety of embodiments described herein are set forth in this application. Any specific details of the embodiments are used for demonstration purposes only, and no unnecessary limitation or inferences are to be understood therefrom.

Before describing in detail exemplary embodiments, it is noted that the embodiments reside primarily in combinations of components related to the system. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In general, the embodiments described herein relate to systems and methods for using AI-enabled automated volumetry of cardiovascular structures. In some embodiments, a system according to the invention is directed to an Al-enabled software module for automated measurement of cardiac chamber volume and left ventricular wall mass that works on non-contrast CT scans, which can be cardiac scans and/or full chest scans. In one embodiment, non-contrast, gated and non-gated, chest CT scans can be used. In some embodiments, the system can identify patients at high risk for developing an adverse health condition, such as AF and heart failure, based on cardiac chamber volume. Embodiments of the systems and methods can facilitate identifying asymptomatic patients at risk of developing atrial fibrillation (AF) and heart failure (HF) based on enlarged left atrium (LA), for example. In one embodiment, the inventive systems and methods can facilitate estimating the volumes of left atrium (LA), left ventricle (LV), right atrium (RA), right ventricle (RV), and left ventricle wall (LVW).

Known systems and methods for measuring the volume of cardiac chambers use contrast-enhanced CT scans, which require more radiation, longer scan times, carry a risk of contrast-induced nephrotoxicity, and demand a higher level of professional staff during the scan. Measurements facilitated by the methods and systems disclosed herein are well correlated with cardiac chambers volumetry measurement using contrast-enhanced CT scans as well as contrast-enhanced cardiac magnetic resonance imaging (CMRI). Measurements, obtained by the inventive methods disclosed, in cardiac CT scans are well correlated with measurements, obtained by the inventive methods disclosed, in full-chest CT scans.

Known methods cannot facilitate detection of asymptomatic individuals at high risk of developing AF or HF based on non-contrast CT scans. The inventive methods and systems disclosed herein can detect asymptomatic individuals at high risk of developing AF or HF based, at least in part, on enlarged left atrium and other cardiac chambers.

Embodiments of the system can provide advantages over existing systems and methods including the potential to reduce or eliminate the need for hospitalization, improve patient quality of life, facilitate patients’ ability to manage their own care (such as through self-directed personal assistance), or establish long term clinical efficiencies.

CHARGE-AF is the most widely referenced epidemiological tool for prediction of AF. Similarly, BNP (brain natriuretic peptide) is the most widely used epidemiological tool for prediction of HF. Embodiments of the inventive systems and methods disclosed herein can outperform CHARGE-AF and BNP for prediction of AF and HF respectively. Adding the volumetry estimates, obtained through the inventive systems and methods disclosed here using cardiac CT scans (such as coronary artery calcium (CAC) scan, or lung cancer screening CT scans), makes it very attractive for population health implementation and primary prevention strategies of the leading cause of death and disability in the United States, cardiovascular disease.

Embodiments of the system include an Al-enabled, automated measurement of cardiac chambers that works on CAC scans and identifies individuals at high risk of AF and HF based on enlarged left atrium and other cardiac chambers.

Over 80 million CT scans are performed in the United States each year. Among them, 95% are non-electrocardiogram (ECG)-gated, low-dose chest screening CT scans for lung cancer screening. Currently, no screening tool is available for identifying patients at high risk of AF or/and HF. CAC scoring represents only a small fraction of all the information available in non-contrast cardiac CT scans.

A digital health screening tool that works on both on ECG-gated cardiac scans and non-gated full lung scans is desirable because it can be used with any coronary artery calcium and lung cancer screening CT scans and provide benefits without additional costs.

Currently, manual segmentation and delineation of one heart with attached great arteries by a well-trained radiologist or cardiologist takes about 30 minutes. However, in some embodiments of the invention disclosed here, a convolutional neural network (CNN) and a vision transformer model (such as U-Net/vision transformer) system can outperform human experts in cardiac segmentation. This segmentation takes less than about 5 seconds in some embodiments.

In certain embodiments, the system detects discrepancies between the myocardium and blood pool that are imperceptible to the human eye. This is a sea change to current interpretation and analysis of cardiovascular structures in a routine CAC or lung cancer screening. Currently, such an analysis is not practiced in clinical.

The Al-enabled approach can add significant values to currently known cardiovascular and lung cancer preventive care, and can reduce healthcare resource disparities by making such an effective, intelligent auxiliary diagnostic tool available through a Software-as-a-Service (SaaS) implementation. In some embodiments, cloud infrastructure and APIs can facilitate implementation of the system as a medical SaaS product to hospitals and imaging centers. Embodiments of the system can be implemented on cloud-based web and mobile app platforms.

In one embodiment, the system can be validated against, for example, a pool of cardiac MRI cases. In some embodiments, the system can be validated against coronary CT angiography cases (for example, 131 never before seen cases). In certain embodiments, the system can be validated against ECG-gated cardiac and non-gated lung scans. Embodiments of the system can use CAC scans and/or lung cancer screening scans, providing benefits without additional imaging cost and/or radiation.

Embodiments of the system can quantify the volume of each cardiac chamber including left atrium (LA), left ventricle (LV), right atrium (RA), right ventricle (RV), LV wall mass, aorta and pulmonary artery from non-contrast CT scans.

Cardiac chamber sizes and left ventricular mass measured by embodiments of the system disclosed here can facilitate predicting future AF, stroke, and HF. Embodiments of the system can use, for example, chest CT scans (either CAC or lung cancer screening scans).

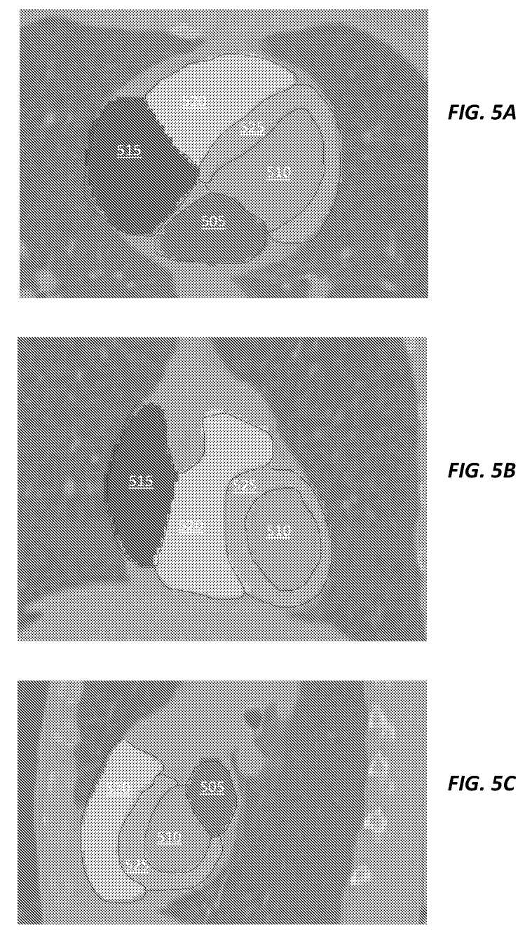

In some embodiments, the system can include a deep learning model to facilitate obtaining accurate measurements of the volume of cardiac chambers and the left ventricular mass from standard, non-contrast chest CT scan images. In some embodiments, the process takes about 5-15 seconds (FIGS. 5A-5C).

Currently, cardiac CT scans are used to image coronary arteries and echocardiography/MRI is used for heart size and hypertrophy evaluation. Embodiments of the system can use one low-cost, reliable technique to assess both aspects of cardiac pathology without subjecting a patient to additional radiation and contrast agents, which in some patients cause nephrotoxicity.

Embodiments of the methods and system disclosed here can substantially shift the role of the calcium score scan, a technique that has remained unchanged (in terms of data and analysis) for about thirty years. Adding volumetric information to the calcium score data can cause the combined volumetry/LVH/calcium score measurements be the most accurate imaging predictor of cardiovascular events available, and possibly the standard of care for preventive cardiology including subclinical coronary artery disease, HF, and detecting pre-AF cases for stroke prevention.

Cardiovascular risk stratification has largely stalled post CAC. While improved calibration and application of CVD risk calculators has occurred, multiple attempts to improve risk prediction with biomarkers or contrast imaging (i.e., CTCA) have not shown benefit with acceptable cost-effectiveness.

Accurate assessment of cardiac chamber sizes, LV mass, aortic size and calcifications, can improve cardiac risk prediction over standard risk equations. A substantial shift in accuracy can move clinical practice from purely Framingham-inspired (cholesterol, BP, smoking) to a combined imaging and risk factor/biomarker approach, changing the paradigm of clinical risk assessment.

Embodiments of the system can impact the conducting of clinical trials for prevention of symptomatic HF and AF. For example, by selecting the top 1 or 5 percentiles of LA size measured by the system disclosed here in a large scale clinical trial, pharmaceutical initiatives can effectively conduct therapeutic intervention trials with a reasonable and affordable sample size.

Results can be visually inspected, and volumetry compared corresponding MRI data. Characteristics of the CT scans resulting in poor volume estimates can be examined. The fine-tuning adjustment of the volumetry algorithms can be performed with selection of the best algorithm and rules to maximize correlation and validation.

In one embodiment, contrast enhanced cardiac CT scans and non-contrast cardiac CT scans can be obtained as part of coronary CT angiography to transfer segmentations of cardiovascular structures from contrast enhanced images to non-contrast images. In some embodiments, the images can be taken from the same patient within minutes and both are ECG gated; hence, the images can be fully registered and the contrast enhanced areas of each cardiovascular structure can be overlapped with the corresponding area in the non-contrast images of the same patient. Since this image is from the same examination, the image is well aligned with the contrast enhanced image.

In one embodiment, if a minor misalignment is detected a human expert can correct the segmentations in the non-contrast images before training. In some embodiments, after a transfer of segmentations, a UNET deep learning system can be used for training an artificial intelligence (Al) model. In certain embodiments, iterative training can be implemented based on human supervised correction of mistakes made by the AI model and inputting the corrected segmentations into the AI model for enhanced training.

In some embodiments, manual editing of segmentation may be required as part of supervised learning. Similarly the system can be improved by patching the model with rules to handle issues related to image acquisition. In some embodiments, a calibration factor can be used to reduce noise effects and CT based LV mass measurements versus MRI. Outputs can be inspected and case failures, and pursue iterative enhancement.

Cardiac chamber volumes are typically measured clinically with ultrasound (echocardiography) or MRI, which are generally considered as the reference standard for clinical care but on a population health level they fall short. As screening tools, echocardiography can be highly operator dependent, and MRI can be excessively high cost and time consuming. More importantly, they cannot detect CAC and are not suitable for lung cancer screening, neither is cardiac CT angiography (ECG gated CT with contrast injection).

Embodiments of the system correlate with MRI for the measurement of LA size, LV end diastolic size, RV end diastolic size and LV mass. Embodiments of the system can delineate the left ventricular wall, outer myocardium, and right ventricle.

One potential limitation of standard CT and calcium score methods is that mid diastole, rather than end diastole is measured. For example, mid diastolic volumes can be converted to end-diastolic measurements with certain ML interpolation, with excellent accuracy. Various machine learning and deep learning and image processing tools can be used to maximize the performance of the system.

The system was used in analyses of AF and HF cumulative incidences with LA and LV volumes based top percentiles. The results show a strong predictive value for LA and LV sizes for prediction of high-risk pre-AF and pre-HF patients who are unaware of their future risk.

Risk factors including LV hypertrophy, which is independent of coronary calcification and can standalone, can be more powerful predictors of HF events than CAC. However, cardiac MRI for this purpose is prohibitively expensive. Echocardiography is also more expensive than the system and requires 1:1 allocation of sonographers. Conversely, a non-contrast CT scan is inexpensive, non-invasive, can be performed in less than 5 minutes, and is highly automated. The system can improve cardiovascular risk assessment and provide a CVD screening triad for CAD, HF, and AF.

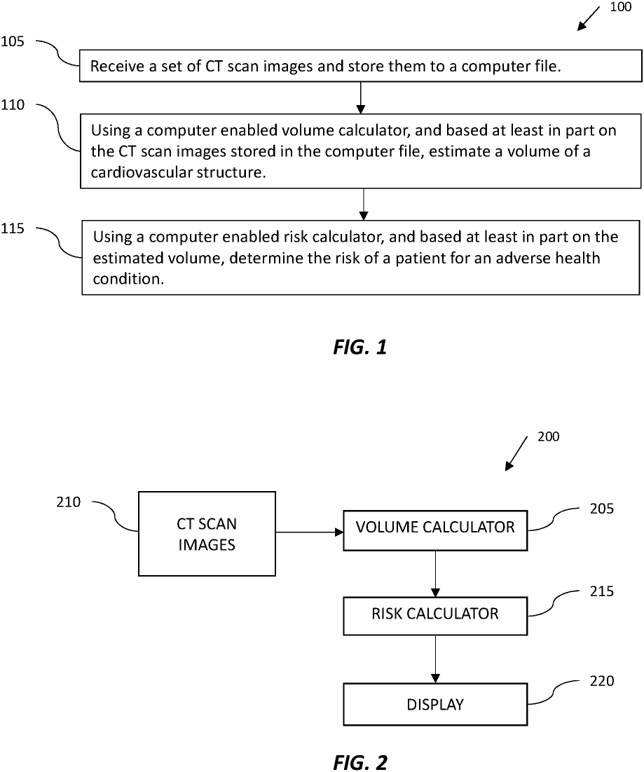

Referencing FIG. 1, method 100 of determining a risk of a patient for an adverse health condition according to one embodiment of the invention is described. At step 105, a set of CT scan images is received and stored in a computer file. In some embodiments, image processing can be applied to the CT scan images to facilitate improving the accuracy of estimating a volume of a cardiovascular structure. At step 110, a computer enabled calculator can be used to estimate a volume of a cardiovascular structure. A suitable computer enabled calculator can be an artificial intelligence (Al) model trained to segment cardiovascular structures and to estimate cardiovascular structure volumes. The AI model can include a deep learning model, machine learning model, and/or rule-based assessment. In some embodiments, the AI model can be trained with contrast enhanced CT scan images. In certain embodiments, the AI model can convert contrast enhanced CT scan images into non-contrast enhanced CT scan images and vice versa. At step 115, a computer enabled risk calculator can be used with the estimated volume to determine a risk of a patient for an adverse health condition. In one embodiment, a computer enabled risk calculator can be a multivariate risk analyzer based, at least in part, on heart failure incidence risk and ROC AUC data. Correlations between cardiac structure volume estimates and heart failure outcome can be used to configure a suitable risk calculator, which can be a multivariate risk calculator.

FIG. 2 illustrates system 200 for facilitating determining a risk of patient for an adverse health condition according to one embodiment of the invention disclosed here. System 200 can include volume calculator 205. In some embodiments volume calculator 205 includes an AI model configured to facilitate determining a volume of a cardiovascular structure. In certain embodiments, system 200 can include CT scan images 210, which can be stored in a computer memory, for example. CT scan images can include images obtained from CT scans, for example, contrast enhanced CT scans, non-contrast enhanced CT scans, ECG-gated cardiac CT scans, non-gated cardiac CT scans, non-gated full chest CT scans, low dose lung cancer screening CT scans, and a combination of contrast enhanced and non-contrast enhanced chest CT scans. In some embodiments the cardiovascular structure can be, for example, left atrium (LA), left ventricle (LV), left ventricular wall (LVW), right atrium (RA), right ventricle (RV), aorta, and/or pulmonary artery.

System 200 can, in one embodiment, include risk calculator 215 configured to take as input the estimated volume of the cardiovascular structure and to determine, based at least in part on the estimated volume, a risk of a patient for an adverse health condition. In certain embodiments, the adverse health condition can be, for example, atrial fibrillation (AF), heart failure (HF), stroke, cerebrovascular events, chronic obstructive pulmonary diseases (COPD), emphysema, ischemic heart disease, cardiovascular mortality, and all-cause mortality. In some embodiments, the risk is determined based on the estimated volume and taking into account other variables, such as patient’s age, gender, height, weight, body surface area, body mass index, and ethnicity, for example. In some embodiments, the estimated volume can be used with one or more health related variables to enhance the prediction model, resulting in a multivariate composite index of health to better determine the risk of future adverse health conditions. In certain embodiments, the one or more health related variables can be, for example, blood pressure, heart rate, blood oxygenation, blood tests, medications, and other patient medical data.

In certain embodiments, system 200 can include display 220 configured for displaying the cardiovascular structure, the estimated volume, and/or a graphic representation of the risk determined by risk calculator 215. In one embodiment, computer enabled display 220 can be on a mobile application or a web application that can be used by patients and/or care providers. In some embodiments, computer enabled display 220 can be on a desktop application run on premises to, for example, avoid patient data security concerns.

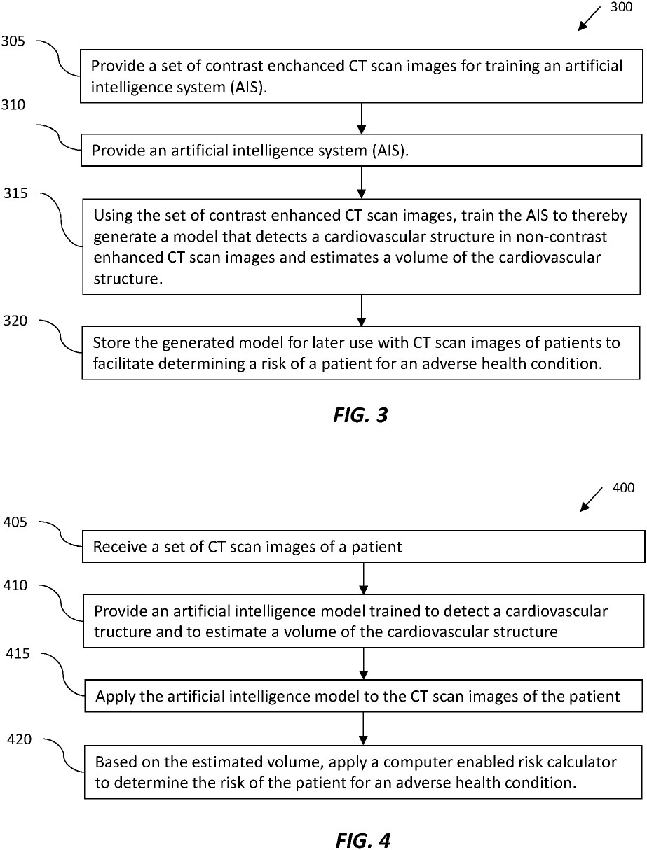

FIG. 3 illustrates method 300 of generating an artificial intelligence model for facilitating determining a risk of patient for an adverse health condition according to embodiments of the invention disclosed here. At step 305, a set of CT scan images can be provided for training an artificial intelligence system (AIS). Preferably, the CT scan images are processed, through suitable images processing algorithms, to optimize the training. At step 310 an AIS can be provided. The AIS can be, for example, a computer enabled and implemented convolutional neural network (CNN). One such CNN can be, for example, U-Net. At step 315, using supervised learning, for example, the AIS can be trained to detect/segment cardiovascular structures from the set of CT scan images. In some embodiments, using supervised learning (for example), the AIS can be further trained to estimate a volume of a cardiovascular structure. As a result of the training a model is generated that can be applied to CT scan images of specific patients at a later time. At step 320, the model generated by the training is stored for later use to facilitate determining a risk of a specific patient for an adverse health condition.

FIG. 4 illustrates method 400 of applying an artificial intelligence model for facilitating determining a risk of a patient for an adverse health condition according to embodiments of the invention disclosed here. At step 405, a set of CT scan images of a specific patient can be received. At step 410, an AIS model trained to detect cardiovascular structures and to estimate a volume of a cardiovascular structure can be provided. At step 415, the AIS can be applied to the set of CT scan images to determine the volume of at least one cardiovascular structure. At step 420, based at least in part on the estimated volume, a computer enabled risk calculator can be applied to determine the risk of the patient for an adverse health condition.

5A-5C show an example of segmentations for cardiac chambers volume measurements performed by, and according to, embodiments of the inventive methods and systems disclosed herein. Referencing FIG. 5A, in one embodiment system 200 can output to display 220 an axial view of cardiovascular structures segmented as LA 505, LV 510, RA 515, RV 520, and LV mass 525. For clarity, the various cardiovascular structures shown in FIGS. 5A-5C are outlined; however, in some embodiments the cardiovascular structures can be displayed in suitable colors (such as red for LA 505, green for LV 510, blue for RA 515, yellow for RV 520, and turquoise for LV mass 525). FIGS. 5A-5C are, respectively, axial, coronal, and sagittal views of cardiovascular structures.

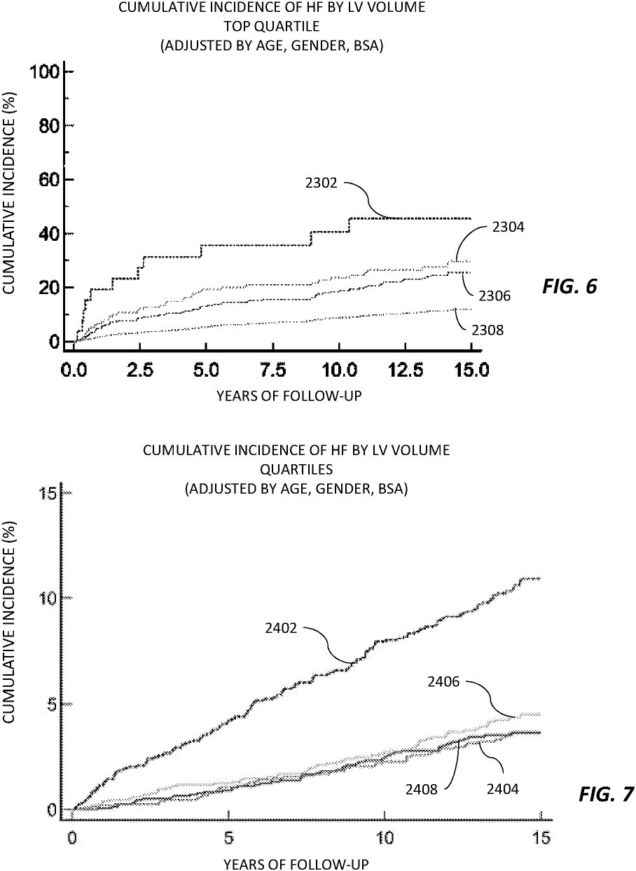

FIGS. 6-9 show cumulative incidence rates of HF and AF using, respectively, LV and LA volume estimates performed by embodiments of system 200 using non-contrast CAC scans. FIG. 6 shows cumulative incidence rates of HF among certain top percentiles of LV volume estimates (N=6,398), as adjusted by age, gender, and body surface area. It is shown top 1% 2302, top 5% 2304, top 10% 2306, and top 25% 2308. FIG. 7 shows cumulative incidence rates of HF among quartiles using LV volume estimates, as adjusted by age, gender, and body surface area. It is shown first quartile 2402, second quartile 2404, third quartile 2406, and fourth quartile 2408.

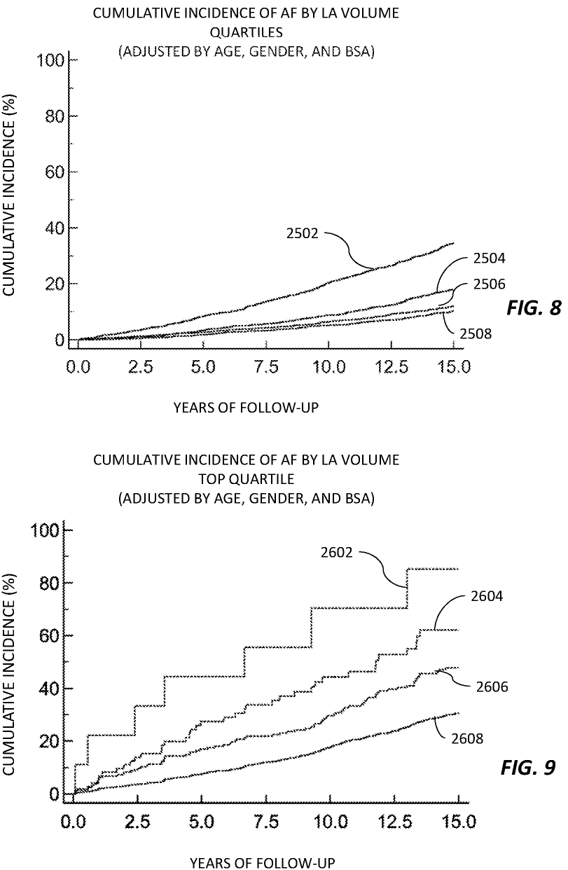

FIG. 8 shows rates of AF among quartiles using LA volume estimates (N=6,334), as adjusted by age, gender, and body surface area. It is shown first quartile 2502, second quartile 2504, third quartile 2506, and fourth quartile 2508. FIG. 9 shows cumulative incidence rates of AF among certain top percentiles using LA volume estimates, as adjusted by age, gender, and body surface area. It is shown top 1% 2602, top 5% 2604, top 10% 2606, and top 25% 2608.

Improving workflow in health care settings, and more specifically for improving the workflow in emergency rooms as well as for reducing patient wait time in health care settings. CT coronary calcium imaging gives direct evidence of coronary artery disease in patents. CT calcium score can be an important factor for predicting the risk of coronary heart disease, with a higher score giving increased 10-year risk.

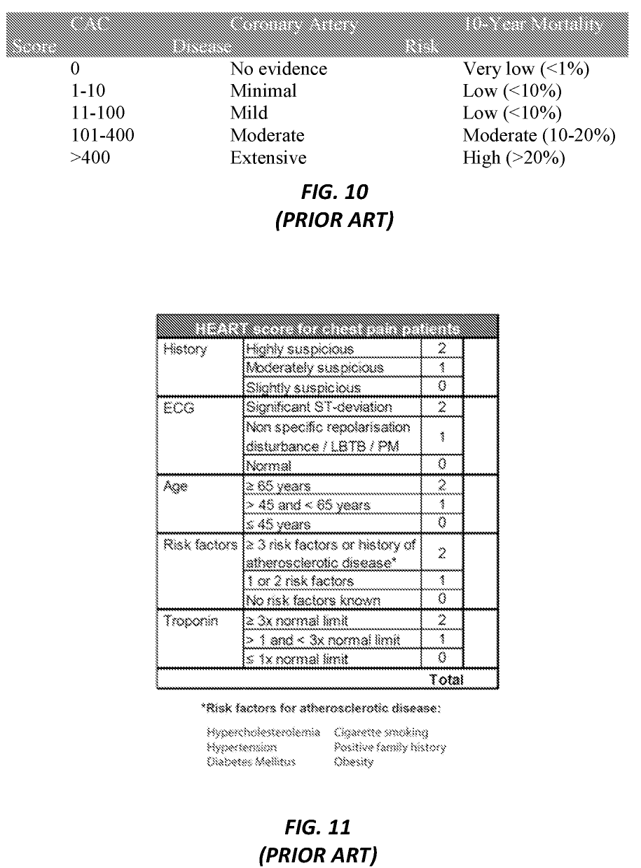

Referencing FIG. 10, the prior art CAC Score table shows varying levels of corresponding Coronary Artery Disease and 10-Year Mortality Risk. Integrating CAC testing very early in evaluating chest pain can appropriately triage and discharge patients who do not require additional testing or invasive procedures. Guideline for the Evaluation and Diagnosis of Chest Pain includes a recommendation for CAC for patients with stable chest pain and no known coronary artery disease (CAD) that are categorized as low risk. CAC testing is reasonable as a first-line test for excluding calcified plaque and identifying patients with a low likelihood of obstructive CAD. FIG. 11 is a prior art HEART SCORE chart illustrating the risk factors involved in calculating heart disease risk.

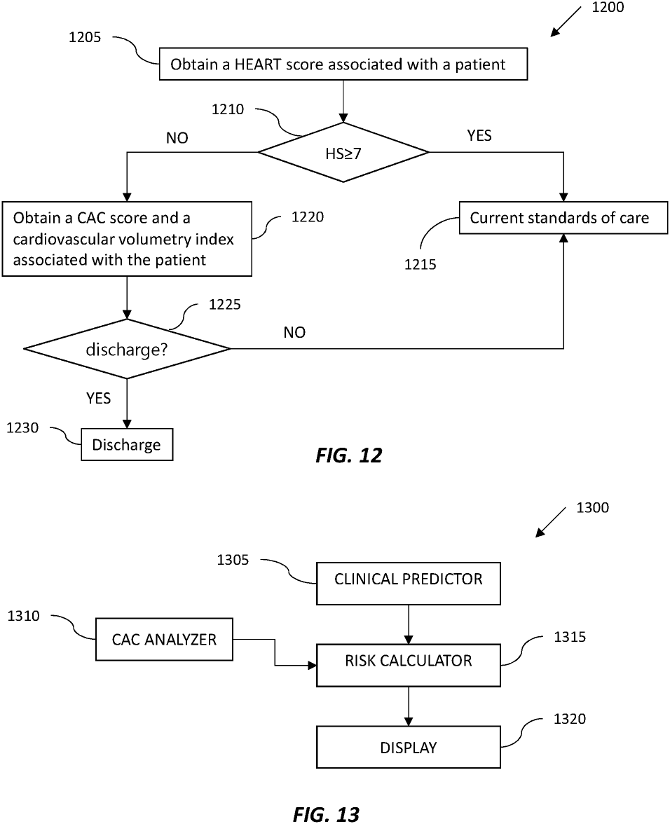

FIG. 12 illustrates method 1200 of assessing risk in person presenting with chest pain. At a step 1205 a HEART score associated with a patient is obtained. At step 1210 it is determined if the HEART score is greater or equal 7. If the HEART score is greater or equal to 7, then the person is directed to current standards of care at a step 1215. If the HEART score is less than 7, then at a step 1220 a CAC score and a cardiovascular volumetry index associated with the person are obtained. The cardiovascular volumetry index can be a multivariate score based, at least in part, in an estimate of a cardiac structure volume, which volume can be obtained by, for example, using the inventive systems and methods disclosed herein. Based on the CAC score and the cardiovascular volumetry index, a recommendation is made at step 1225 of whether to discharge the person. If the recommendation is to not discharge the person, the person is directed to current standards of care 1215. If the decision is to discharge the person, then a recommendation is made to discharge the person at step 1230.

FIG. 13 illustrates system 1300 for expediting the risk assessment of a patient presenting with chest pain. In one embodiment, system 1300 includes clinical predictor 1305 configured to determine a first risk score associated with the patient. In some embodiments clinical predictor 1305 can be a computer enabled, automated module that produces a HEART score based on patient data. System 1300 can include CAC analyzer 1310 configured to provide a CAC score and a cardiovascular volumetry risk index based on CT scan images associated with the patient. In certain embodiments system 1300 can include risk calculator 1315 configured to: determine a second risk score based on the CAC score and the cardiovascular volumetry risk index; and based at least in part on the second risk score, to automatically provide a recommendation on whether to discharge the patient. In some embodiments, system 1300 can include display 1320 suitably configured to display the first risk score, HEART score, CAC score, volumetry data (including digital representations of cardiac structure volumes), and/or cardiovascular risk index.

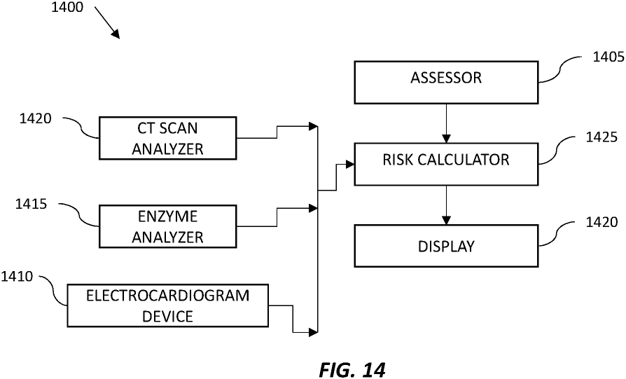

FIG. 14 illustrates system 1400 for triaging a person with chest pain, such as a person presenting at an emergency department of a hospital. In one embodiment, system 1400 can include assessor 1405 configured to assess a history of cardiovascular risk factors and symptoms of chest pain of a person; electrocardiogram device 1410 configured to obtaining electrocardiogram related data from the person; enzyme analyzer 1415 configured to determine troponin enzyme levels associated with the patient; and CT scan analyzer 1420 configured to provide a CAC score and a cardiovascular volumetry risk index based on CT scan images of the person. In some embodiments, system 1400 can include computer enabled risk calculator 1425 configured to: generate a first risk score for the person based on electrocardiogram, troponin level, age of the patient, other risk factors, and/or history of symptoms; based at least in part on the first risk score, assign to the person a risk category (for example, low, intermediate, and high); generate an order for the person to undergo a non-contrast cardiac CT scan if the risk category assigned to the person is low or intermediate; based at least in part on a CAC score and a cardiovascular volumetry risk index determined by CT scan analyzer 1420, determine a second risk score for the person; and based at least in part of the second risk score, recommend discharge of the patient from the emergency department if the second risk score is below a predetermine threshold.

In one embodiment, CT scan analyzer 1420 uses artificial intelligence to calculate a CAC score. In some embodiments, the artificial intelligence used to calculate a CAC score can include: detecting features from at least one or more portions of CT scan images, electrocardiogram related data, and/or troponin enzyme level data that fall within each of one or more temporal windows; identifying patterns in the detected features based on one or more of the following models: a classification model and a regression model; and using the identified patterns, calculate, a probability of whether the identified patterns correspond to a CAC score of a person.

In certain embodiments, system 1400 can be configured to generate one or more additional reports from CT scan results covering one or more subject matters from the following group: lung nodules, emphysema score, cardiac and aortic sizes, pericardial fat, fatty liver, and bone density. In one embodiment, system 1400 is configured so that a CAC score of zero in patients younger than 40 years is not used for discharge due to higher prevalence of non-calcified coronary plaques, and wherein in these younger populations either repeated HEART score or measurement of carotid wall thickness also called carotid intima-media thickness (CIMT) may be used for maximizing accuracy and reducing potential liability of missing coronary events.

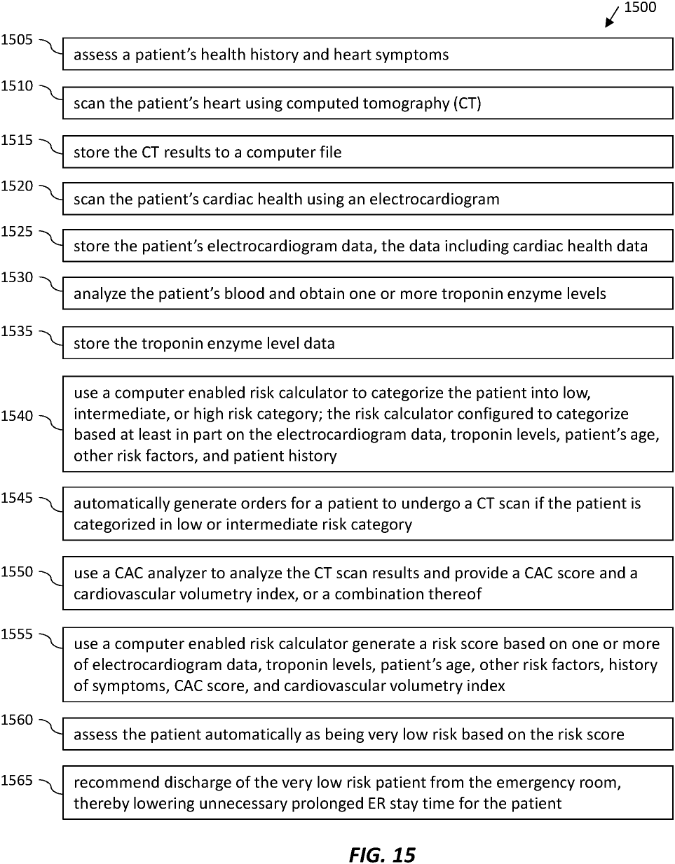

FIG. 15 illustrates method 1500 of assessing patients presenting with chest pain. In one embodiment, method 1500 includes assessing a patient’s health history and heart health symptoms 1505; scanning the patient’s heart using computed tomography (CT) 1510; storing the CT results to a computer file 1515; scanning the patient’s cardiac health using an electrocardiogram 1520; storing the electrocardiogram generated data resulting from said scanning, the data comprising cardiac health data 1525; analyzing the patient’s blood and obtaining one or more troponin enzyme levels of the patient’s blood 1530; storing the troponin enzyme level data resulting from said testing 1535; use a computer enabled risk calculator to categorize the patient into low, intermediate, or high risk category; the risk calculator configured to categorize based at least in part on the electrocardiogram data, troponin levels, patient’s age, other risk factors, and patient history 1540; automatically generating orders for a patient to undergo a CT scan if the patient is categorized in low or intermediate risk category 1545; using a CAC analyzer to analyze the CT scan results and provide a CAC score and a cardiovascular volumetry index, or a combination thereof 1550; using a computer enabled risk calculator generate a risk score based on one or more of electrocardiogram data, troponin levels, patient’s age, other risk factors, history of symptoms, CAC score, and cardiovascular volumetry index 1555; assess the patient automatically as being very low risk based on the risk score 1560; recommending discharge of the very low risk patient from the emergency room, thereby lowering unnecessary prolonged ER stay time for the patient 1565.

In some embodiments, method 1500 can include using artificial intelligence to calculate a CAC score. In certain embodiments, method 1500 can include generating additional reports from the CT scan results are generated covering one or more subject matters from the following group: lung nodules, emphysema score, cardiac and aortic sizes, pericardial fat, fatty liver, and bone density. In one embodiment, method 1500 can include generating reminders to be communicated to the patient for follow up services.

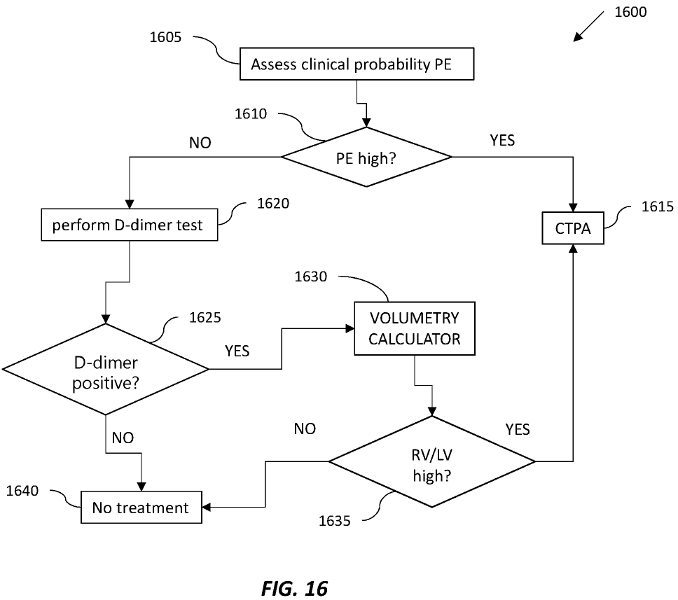

Referencing FIG. 16, method 1600 of assessing patient’s risk of pulmonary embolism (PE) is illustrated. PE can show chest pain symptom and can be caused by blood clots in pulmonary arteries. A blood clot puts pressure on the RV and causes it to become larger. Therefore, cardiovascular volumetry of RV versus LV can be useful in flagging potential PE. Although PE is currently detected with contrast enhanced pulmonary angiography, cardiovascular volumetry, as performed by th inventive systems and methods, can provide a ratio of RV over LV (RV/LV). In some embodiments, if RV/LV is greater than 2-2.5, the patient can be flagged for potential PE. Embodiments of the inventive systems and methods disclosed herein can provide the same on contrast enhanced CT angiography of pulmonary arteries as well.

In one embodiment, method 1600 includes assessing the clinical probability of PE for a patient 1605. The patient may have chest pain symptoms. At step 1610 it is determined if there is a high probability of PE. If there is a high probability of PE, then a Computed Tomography Pulmonary Angiogram (CTPA) can be ordered 1615. If the probability of PE is not high (that is, low or intermediate, for example), then a D-dimer test is performed 1620. At a step 1625 it is determined whether the D-dimer test is positive 1625. If the D-dimer test is not positive, then no treatment is recommended 1640. If the D-timer is positive, then cardiovascular volumetry can be performed 1630. In one embodiment, cardiovascular volumetry can be performed by, for example, a volume calculator in accordance with the systems and methods disclosed herein. Left ventricle (LV) and right ventricle (RV) volumes are determined. In some embodiments a ratio of RV to LV is calculated 1630. If the RV/LV ratio is high 1635, a CTPA can be ordered. If the RV/LV is low 1635, no treatment is ordered 1640.

Some embodiments of the inventive systems and methods can include the Auto-CAC^{T″′} scan, a non-contrast low-dose CT scan of the chest that provides a rapid coronary artery calcium (CAC) score using the onsite CT machine and cloud-based supervised AI powered by HeartLung^{TM} . The AI used can be any AI known to those skilled in the art and the steps can be performed on any suitable cloud including network based clouds such as those found on the internet. In many of these embodiments, CT coronary calcium imaging is used to automatically calculate a CAC score. In some embodiments, the Auto-CAC systems and methods using a HeartLung™ branded computing device application can perform the CAC at any time and deliver a cardiac volumetry index (that is, score) to a health care professional and/or a patient within a short period of time (for example, 30 minutes). The automatically generated score can assist these health care professionals such as ED physicians in determining if a patient can be safely discharged.

These described embodiments can help reduce the risk of misdiagnoses and inadvertent discharges of patients with Acute Coronary Syndrome (ACS). They can also help reduce the number of patients sent to additional (and unnecessary) testing or invasive procedures. The embodiments can also increase the rapid and accurate diagnosis of patients suitable for discharge home including those with an excellent prognosis. They can also help increase patient flow through the ED, allowing for more efficient use of time and resources.

In these embodiments, using a low-dose chest CT scan (e.g. with the described Auto-CAC or auto scoring embodiments] and hs-cTn enzyme level information in a patient, within a short period of time (30 minutes, for example), can help rule out the presence of CAD, and any subsequent need for emergent CTA. In many embodiments, Myocardial infarction (MI) can be ruled out in patients with troponin concentrations <5 ng/L at presentation, or if delta <3 ng/L and remain below the 99th centile after 3 hours.

Additionally, combining CAC with the initial troponin from the HEART Score can lead to greater than 99% NPV with a number of benefits, including the systems and methods being cost-effective, easy to perform (no patient preparation required and procedures completed in a single breath hold), no contrast required, widely available, easy to interpret results, and a higher NPV of CAC = 0 compared to other methods known. These embodiments use CAC testing with subsequent interpretation of the results that can be completed rapidly and anytime including outside of regular clinic hours. Another benefit is that these embodiments can be used for prompt evaluation which can lead to a quicker discharge from ED or other health care setting when appropriate. A further benefit of these embodiments is that the radiation dosage used is minimal, for example a median dose for a low-dose CAC is 1.1 mSv.

The described embodiments are cost-effective and covers a larger population than hs-cTn diagnostics alone. At a low cost and minimal time requirement, in many cases a patient can be discharged home after a low-to- intermediate CAC score and a negative troponin level, along with a report to share with a medical professional. This compares to much higher costs for a full troponin series coupled with many hours of waiting for testing and test results. This can help reduce the cost for health care, as well as reduce low-risk patient emergency department visit time, which allows health care providers to allocate more time to complex, higher-revenue patient care.

Embodiments also include the use of an app or software for any computing platform which can provide a number of additional benefits, such as a 30-day post-discharge self-report triage and reminders for scheduling visits with PCPs.

Additional tracking of patients with suitable records can be maintained including the use of the HeartLung™ application with discharged patients. Other tracking such as leadership in CAC with ACC/AHA guidelines-based care pathway can also be incorporated into the ED or other health care setting procedures. Additional business intelligence data from HeartLung ™ app dashboard, such as analytics and usage of the app, can guide hospital management with ED-related key performance indicators (KPI). For example, both patients and their relatives can benefit from the incorporation of new guidelines based on this data by resulting in shorter ER stays. Embodiments can also include one or more full chest CT reports for EDs without the need for overnight 24/7 tele-radiology.

Some embodiments include participation in the HeartLung™ (Al-Powered) Auto-10™ program which results in ten actionable patient-friendly reports from the same chest CT scan covering lung nodules, emphysema score, cardiac and aortic sizes, pericardial fat, fatty liver, bone density and other actionable information. These additional results can be provided to patients and their providers at low or cost and with no added radiation exposure to the patient.

1. A method of expediting the risk assessment of a patient presenting with chest pain, the method comprising:
determining a first score based at least in part on data associated with the age and a troponin level of the patient;
if the first score is below a predetermined risk level, obtaining a CAC score and a cardiovascular volumetry risk index associated with the patient; and
based on the CAC score and the cardiovascular volumetry risk index, making a recommendation associated with discharging the patient.

2. The method of claim 1, wherein determining a first score comprises determining a HEART score.

3. The method of claim 1, wherein obtaining a cardiovascular volumetry risk index comprises obtaining an estimate of left ventricle volume.

4. The method of claim 3, wherein obtaining an estimate of left ventricle volume comprises using an artificial intelligence (Al) enabled volume calculator.

5. The method of claim 4, wherein using an Al enabled calculator comprises using non-contrast enhanced CT scan images as input to the Al enabled calculator.

6. A system for facilitating assessment of patients presenting with chest pain, the system comprising:
a clinical predictor configured to determine a first risk score associated with the patient;
a CAC analyzer configured to provide a CAC score and a cardiovascular volumetry risk index based on CT scan images associated with the patient; and
a risk calculator configured to:
determine a second risk score based on the CAC score and the cardiovascular volumetry risk index; and
based at least in part on the second risk score, to automatically provide a recommendation on whether to discharge the patient.

7. The system of claim 6, wherein the clinical predictor is configured to determine a first risk score based at least in part on a HEART score.

8. The system of claim 6, wherein the CT scan images comprise non-contrast enhanced CT scan images.

9. The system of claim 6, wherein the CAC analyzer is configured to use artificial intelligence (Al) to provide the CAC score and/or the cardiovascular volumetry index.

10. The system of claim 9, wherein the Al is configured to provide the CAC score and/or the cardiovascular volumetry index based, at least in part, on analysis of non-contrast enhanced CT scan images associated with the patient.

11. The system of claim 6, wherein the CAC analyzer is configured to provide the cardiovascular volumetry risk index by, at least in part, obtaining an estimate of left ventricle volume.

12. The system of claim 11, wherein obtaining an estimate of left ventricle volume comprises using an artificial intelligence (Al) enabled volume calculator.

13. The system of claim 12, wherein using an Al enabled calculator comprises using non-contrast enhanced CT scan images as input to the Al enabled calculator.

14. A method of assessing a patient presenting with chest pain, the method comprising:
assessing the patient’s health history and heart health symptoms;
scanning the patient’s heart using computed tomography (CT);
storing the CT results to a computer file;
scanning the patient’s cardiac health using an electrocardiogram device;
storing the electrocardiogram generated data resulting from said scanning, the data comprising cardiac health data;
analyzing the patient’s blood and obtaining one or more troponin enzyme levels of the patient’s blood;
storing the troponin enzyme level data resulting from said testing;
using a computer enabled risk calculator categorize the patient into low, intermediate, or high risk category, wherein the risk calculator is configured to categorize the patient, based at least in part on the electrocardiogram data, troponin levels, patient’s age, other risk factors, and patient history;
automatically generating orders for a patient to undergo a CT scan if the patient is categorized in low or intermediate risk category;
using a CAC analyzer to analyze the CT scan results and provide a CAC score and a cardiovascular volumetry index;
using a computer enabled risk calculator to generate a risk score based on the CAC score and the cardiovascular volumetry index;
assessing the patient automatically as being very low risk based on the risk score; and
recommending discharge of the very low risk patient from the emergency room, thereby lowering unnecessary prolonged ER stay time for the patient.

15. The method of claim 14, wherein using a CAC analyzer comprises using artificial intelligence (Al) to provide the CAC score and/or the cardiovascular volumetry index.

16. The method of claim 15, wherein using Al comprises using non-contrast enhanced CT scan images as input to the Al.

17. The method of claim 15, wherein using Al to provide the cardiovascular volumetry index comprises estimating a volume of a left ventricle.

18. The method of claim 15, further comprising using the Al to estimate a right ventricle (RV) volume and a left ventricle (LV) volume, and flagging patients for potential pulmonary embolism (PE) if the RV volume to LV volume ratio is above a predetermined normal ratio.

19. A method of assessing a patient’s risk of pulmonary embolism (PE), the method comprising:
assessing the patient’s health history and evaluating clinical likelihood of PE;
measuring the patient’s D-dimer;
automatically generating orders for the patient to undergo a CT scan if the patient is categorized in a low or an intermediate risk category;
using an Al-enabled cardiovascular volume calculator to obtain a right ventricle (RV) volume and a left ventricle (LV) volume;
determining a RV volume to LV volume ratio; and
determining a PE risk based at least in part on the RV volume to LV volume ratio.

20. The method of claim 19, wherein the CT scan comprises a non-contrast enhanced CT scan.
